(19) United States
(12) Patent Application Publication
(10) Pub. No.: US 2008/0041150 A1
(43) Pub. Date: Feb. 21, 2008

(54) MEASURING DEVICE FOR DETECTING PRODUCT STRIPS ON A FABRIC

(76) Inventors: Kerstin Lohr, Krefeld (DE); Bernhard Funger, Krefeld (DE); Andreas Piechowiak, Krefeld (DE)

Correspondence Address: KENYON & KENYON LLP, ONE BROADWAY, NEW YORK, NY, 10004, US

(21) Appl. No.: 11/836,494

(22) Filed: Aug. 9, 2007

(30) Foreign Application Priority Data
Aug. 16, 2006 (DE) 10 2006 038 436.9

(51) Int. Cl. G01N 33/36; G01N 33/34; G01R 27/26

(52) U.S. Cl. 73/159; 324/686; 340/815.4

(57) ABSTRACT

A measuring device for detecting product strips on a fabric, e.g., a paper-including or textile-including fabric, includes a sensor, that records optical, magnetic or electrical properties of the fabric, which converts the recorded property to a measuring signal, a device for establishing a signal threshold value, and a signal device which emits an optical and/or an acoustical signal when a change in the measuring signal beyond the threshold value is taking place. Using this measuring device, strip positions and strip widths may be recorded of the strips applied to the fabric which are not visible to the human eye.

### CROSS-REFERENCE TO RELATED APPLICATIONS

The present application claims priority to Application No. 10 2006 038 436.9, filed in the Federal Republic of Germany on Aug. 16, 2006, which is expressly incorporated herein in its entirety by reference thereto.

### FIELD OF THE INVENTION

The present invention relates to a measuring device for detecting product strips on a fabric.

### BACKGROUND INFORMATION

Fabric includes paper fabric or textile-containing fabric. Nonwoven fabric or fleece layers that are used in hygienic articles also belong to the category of textile-containing fabrics. Such nonwoven fabric layers often have strips of a product applied in liquid form onto the nonwoven fabric layer, whose positions cannot be detected with the naked eye after drying. As an example, diapers whose nonwoven fabric layers are provided, in the area where the impingement of body fluids is to be expected, with a strip that resorbs wetness, in order to accomplish carrying away the body fluid from the surface of the nonwoven fabric layer to a deeper layer that absorbs the body fluid.

Since there is a large requirement for nonwoven fabric layers finished in that manner, a plurality of strips of the product are simultaneously applied over the width of an advancing nonwoven fabric web, during industrial production. After the application, and possibly after further steps for fixing, drying, etc., of the product on the nonwoven web, a longitudinal partition of same is performed into strips, whose width corresponds to that of the nonwoven fabric layer required, using a suitable separating device (e.g., a roll slitter).

For error-free production, one has to know both the width of the strips and their position on the nonwoven fabric web, in order to be able to control an application device, such as that described in German Published Patent Application No. 32 09 456, for the purpose of obtaining the desired strip width, and in order to be able to cut the nonwoven fabric layer into individual longitudinal strips at the right places. This control is made especially difficult for nonwoven fabric webs in which the width and/or the position of the applied strip changes in subsequent method steps.

In the case where the product is applied in aqueous solution, as it is in an overwhelming number of cases, a subsequently added drying step is necessary, in which the nonwoven fabric web regularly shrinks and in which, consequently, the position and the width of the strips change. Therefore, to achieve an optimal production result, it is necessary to establish the strip width and the strip position after total completion of the application of the strips and of their fixing that takes place, for instance, by drying. This is currently being done by first making a strip-shaped application of the product in a test run, according to values taken from experience, and subsequently determining the strip positions and the strip widths by manually wetting the nonwoven fabric web that has been provided with the strips. After the strip positions and the strip widths have been ascertained in this manner, the application device and the cutting device are set to achieve the desired width of the strips and the position of the strips on the nonwoven fabric layers.

A disadvantage in this method is that costly laboratory tests are required to determine the strip positions and the strip widths, which are possible exclusively when the nonwoven fabric web is standing still. In addition, the areas of the nonwoven fabric web (more generally, the fabric), at which laboratory tests for the determination of the strip positions and the strip widths are carried out, are unusable for later use. This reduces productivity, and production scrap is created that has to be disposed of appropriately, and this creates additional costs.

### SUMMARY

Example embodiments of the present invention provide a measuring device with the use of which product strips may be detected on a fabric, e.g., a paper fabric or a textile-containing fabric, that cannot be perceived by the human eye.

The device includes a sensor, that detects optical, magnetic or electrical properties of the fabric, which converts the detected property to a measuring signal. The measuring device utilizes the aspect that, in the areas to which the product has been applied, which cannot be perceived by the naked eye, one or more of the named properties of the textile-containing fabric have significantly different values than in the areas in which the product has not been applied. If optical properties of the fabric are to be measured, the measuring device includes a CCD chip, for instance, using which, for example, the intensity is recorded of an electromagnetic radiation, not necessarily in the visible spectrum, that is reflected by a surface of the fabric. For this purpose, the illumination of the surface of the fabric may be performed by a light source that emits electromagnetic radiation of a desired wavelength. It may be integrated into the measuring device. The magnetic properties of the fabric may be measured if the measuring device includes a coil device whose inductance is used for generating the measuring signal, when the fabric is penetrated by magnetic field lines generated by a coil device. However, the measuring device may include a sensor for recording the electrical properties of the fabric, for which the area of the fabric, whose electrical properties are to be measured, may be brought into an electrical field.

The device also includes a device for establishing a signal threshold value, as well as a signal device that emits an optical and/or an acoustical signal when there is a change in the measuring signal beyond the threshold value.

In order to be able to detect the strip positions, the measuring device is moved over the fabric. The signal threshold value is set beforehand such that, in the case of a fabric, onto which no product has been applied, the expected signal is on one side of the threshold value, but that after the product is applied, the measuring signal is on the other side of the threshold value. The transition of the measuring signal from the one side to the other side of the threshold value is indicated optically or acoustically, using the signal device, and that is how the position of the edge of a strip may be established.

The measuring device may include a capacitively working sensor. This may have a plate-type capacitor device which is positioned such that the area in which the property of the fabric is to be recorded, is penetrated by electrical field lines that are generated by the plate-type capacitor device.

For this purpose, an open plate-type capacitor may be provided, whose plates are, for example, arranged side by side in one plane, such that both plates are located on one side of the fabric during the measurement.

If the measuring device includes a capacitively operating sensor, the latter may be operated according to a method such as that described, e.g., in German Published Patent Application No. 41 00 869.

For operating personnel to be able to detect in a simple manner the strip positions and the strip widths when the fabric is scanned, the signal device of the measuring device may include an illumination device and/or a loudspeaker. The position of the edge of a strip may be recorded as a function of the spatial resolution achieved by using the measuring device, which is a function of the size of the area of the fabric used for the property detection, in that, in a simplest case, the operating personnel optically marks the position on the fabric at which the signal appears.

The measuring may include a display for the playback of the threshold value and the signal. Based on this refinement, the operating personnel has available information on the currently set threshold value, which reduces the probability of erroneous measurements caused by a wrongly set threshold value. In addition, based on the size of the measuring signal that may be read off from the display, one may draw a conclusion concerning the coverage of the area, that is, concerning the quantity of the product applied per area, provided the measuring device has been calibrated beforehand with the aid of tests having specified area coverage.

The measuring device may include its own internal energy supply, which makes it particularly suitable for use as a mobile handheld unit.

The measuring device may include a signal output for connecting to an external data processing installation, by which the measuring signals present at the signal output may be stored and, for instance, be recalculated into surface coverage values. The measuring device may be able to be connected to an external data processing installation, if it also includes a connection for an external energy supply, since the measuring device features, because of the saving of the internal energy supply, a particularly low requirement for space, and consequently, its use is facilitated even in the case of tight space conditions.

The measuring device may also be simply integrated into existing systems if the connections for the signal output and the energy supply are provided according to, e.g., the USB standard.

This may be provided as a mobile handheld unit in a first application area, for which the measuring device is supposed to be suitable. This is particularly suitable for recording the strip positions and the strip widths on a fabric even if it is standing still, by manually guiding the measuring device over the fabric and marking those places on the fabric at which the optical and/or the acoustical signal is emitted with the aid of the signal device.

In order, however, to be able to integrate the measuring device into a production sequence in which the fabric runs continuously, it may be configured so that it may be coupled to a displacement-measuring system. Using this displacement-measuring system, the measuring signals are allocated to the respective location of the sensor with respect to the fabric.

The displacement-measuring system may include a device by the use of which the sensor is displaceable transversely to the longitudinal extension of the strips. The device may grip a spindle drive, for example, by which the sensor may be displaced over the fabric. The measuring signal recorded by the sensor may be simply allocated to a position, if the drive of the spindle includes a stepper motor that is controlled by a data processing installation, so that the position of the sensor is established by the executed steps.

However, for the purpose of determining position, the displacement-measuring system may also have a separate displacement gauge having a position signal output, at which there is present a signal that is a function of the sensor position. The position signal output may be connected to the data processing installation for the purpose of correlating the recorded measuring signals with the respective sensor position.

According to an example embodiment of the present invention, a measuring device for detecting product strips on a fabric includes: a sensor configured to detect at least one of (a) optical, (b) magnetic and (c) electrical properties of the fabric, the sensor configured to convert a detected property to a measurement signal; a device configured to establish a signal threshold value; and a signal device configured to emit at least one of (a) an optical and (b) an acoustical signal when a change in the measurement signal beyond the threshold value is taking place.

The fabric may be arranged as at least one of (a) a paper-including fabric and (b) a textile-including fabric.

The sensor may be arranged as a capacitive sensor.

The sensor may include a plate-type capacitor device.

The plate-type capacitor device may include an open plate-type capacitor.

The signal device may include at least one of (a) an illumination device and (b) a loudspeaker.

The measuring device may include a display configured to display the threshold value and the measurement signal.

The measuring device may include an internal energy supply.

The measuring device may include a connection connectable to an energy supply.

The measuring device may include a signal output connectable to an external data processing installation.

The measuring device may include a USB connection connectable to an energy supply and to an external data processing installation.

The measuring device may be arranged as a mobile handheld unit.

The measuring device may be coupleable to a displacement-measurement system.

The displacement-measurement system may include a device configured displace the sensor transversely to a longitudinal extension of a product strip over the fabric.

The displacement-measurement system may include a position signal output at which a signal is present that is a function of a sensor position.

The position signal output may be connected to the data processing installation.

According to an example embodiment of the present invention, a system includes: a measurement device configured to detect a product strip on a fabric; and a displacement-measurement system, the measuring device coupled to the displacement-measurement system. The measurement device includes: a sensor configured to detect at least one of (a) optical, (b) magnetic and (c) electrical properties of the fabric, the sensor configured to convert a detected property to a measurement signal; a device configured to establish a signal threshold value; and a signal device configured to emit at least one of (a) an optical and (b) an acoustical signal when a change in the measurement signal beyond the threshold value is taking place; and

According to an example embodiment of the present invention, a method for detecting product strips on a fabric includes: detecting at least one of (a) optical, (b) magnetic and (c) electrical properties of the fabric; converting a detected property to a measurement signal; establishing a threshold value; and emitting at least one of (a) an optical and (b) an acoustical signal when a change in the measurement signal beyond the threshold value is taking place.

Example embodiments of the present invention are described in more detail below with reference to the appended Figures.

### BRIEF DESCRIPTION OF THE DRAWINGS

FIG. 1 schematically illustrates a measuring device configured as a mobile handheld unit, in a top view onto a fabric.

FIG. 2 schematically illustrates a measuring device, which includes a displacement-measuring system, in a view similar to that of FIG. 1.

FIG. 3 schematically illustrates the measuring device illustrated in FIG. 2 taken from the left.

### DETAILED DESCRIPTION

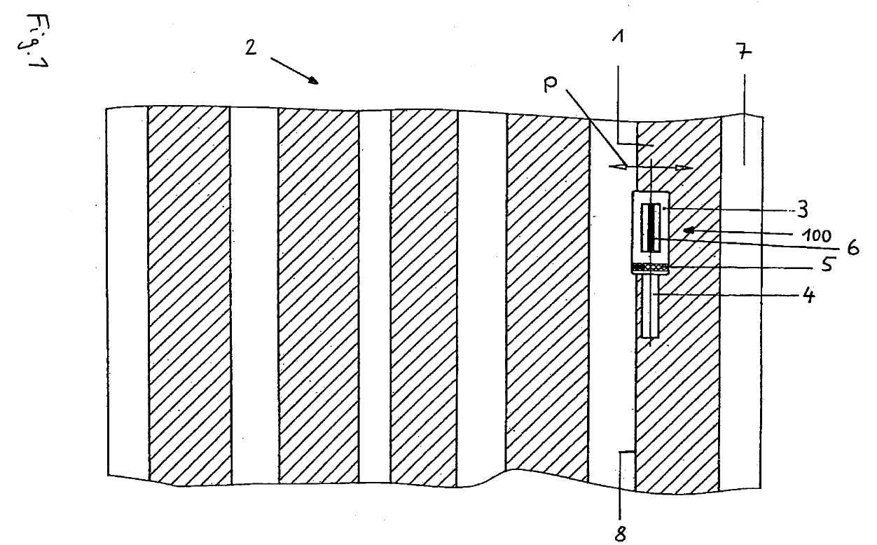

The measuring device designated as 100 in its entirety in FIG. 1 is used to detect product strip 1 on a fabric 2. Product strips 1 may be, e.g., strips of a product applied to fabric 2 in liquid form, and which cannot be detected by the naked eye. If the fabric is a nonwoven layer, for example, which is to be used for hygienic articles such as diapers, the product strips may include a material that confers a wetness resorbing effect on the nonwoven fabric layer.

Measuring device 100 includes a housing 3 that is provided with a handle 4.

A sensor is provided in housing 3, which records properties of the fabric that are influenced by product strip 1 and converts them into a measuring signal.

The sensor may be provided as a capacitively working sensor. It includes a plate-type capacitor device, which is configured such that, in the measuring position of measuring device 100, which is located right up against the fabric, or closely above it, the electrical field generated by the plate-type capacitor device penetrates an area of fabric 2.

The plate-type capacitor device is operated, using an electronic circuit, e.g., in a conventional manner, such as that described in German Published Patent Application No. 41 00 869, which is expressly incorporated herein in its entirety by reference thereto.

Measuring device 100 also includes a device for establishing a signal threshold value and a signal device 5 that emits an optical and/or an acoustical signal when there is a change in the measuring signal beyond the threshold value.

Furthermore, a display 6 is accommodated in the housing which indicates the threshold value as well as the currently recorded measuring signal.

In addition, measuring device 100 has an internal energy supply, which may be provided, for example, in the form of batteries or rechargeable accumulators in handle 4.

In order to ascertain the strip positions and the strip widths, measuring device 100 is displaced by operating personnel e.g., manually, transversely to the longitudinal extension of product strip 1, over fabric 2, as is indicated by double arrow P. In order to obtain reproducible results, the manual displacement of measuring device 100 is made with it in contact with fabric 2.

In the transition from a stripless area 7 to an area furnished with a product strip 1, there is a change in the capacitive properties of the area of fabric 2, that is penetrated by the electrical field of the plate-type capacitor device of measuring device 100. According to empirical tests carried out before the measurement, the threshold value is set such that the measuring signal generated using the sensor in stripless areas is on one side of the threshold value, and in areas furnished with product strips is on the other side of the threshold value.

When the edge of a strip is swept over, there is a transition of the measuring signal from the one side of the threshold value to the other, which activates signal device 5. Consequently, the position of strip edge 8, which has just been swept over, may be marked by the operating personnel.

The measuring device 100 described above may provide that, using it in separation from a production device, the strip positions and the strip widths of product strips 1 may be ascertained. It is particularly suitable for a random sample-like measurement on fabrics 2 that are standing still.

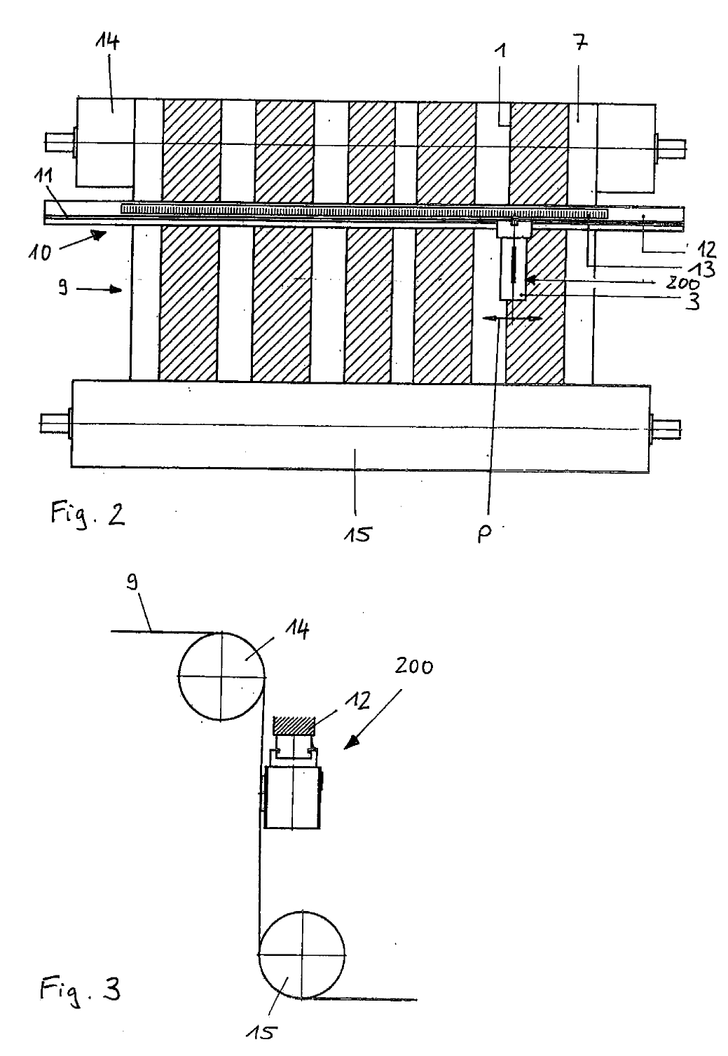

In contrast, the measuring device 200 illustrated in FIGS. 2 and 3 is provided, e.g., for recording the strip positions and the strip widths of product strips 1 in response to a continuously advancing fabric, e.g., a fabric web 9.

In order to do this, measuring device 200 working according to the same measuring principle as measuring device 100 is coupled to a displacement-measuring system 10. It includes a spindle 11 that collaborates with housing 3 of measuring device 200, and a rail device 12 that guides housing 3, so that housing 3 is displaceable over the entire width of fabric web 9 by the rotary operation of spindle 11, transversely to the longitudinal extension of product strips 1, as indicated by double arrow P. The strip positions and/or the strip widths may be recorded by correlating the measuring signal of measuring device 200 with a scaling 13 provided on rail device 12. It is also possible to supply the measuring signal to a data processing installation, which also controls a stepper motor that is provided for the rotary operation of spindle 11, and which may thus allocate the measuring signals to positions of housing 3.

As inferrable from FIG. 3, the positioning of measuring device 200 may take place in an area in which the advancing fabric web is tensioned to form a plane, which may be accomplished by driven guide rolls 14, 15 that are connected before and after measuring device 200 and tension the fabric webs.

### LIST OF REFERENCE SYMBOLS

100, 200 measuring device
1 product strip
2 fabric
3 housing
4 handle
5 signal device
6 display
7 stripless area
8 strip edge
9 fabric web
10 displacement-measuring system
11 spindle
12 rail device
13 scaling
14 guide roll
15 guide roll
P double arrow

What is claimed is:

1. A measuring device for detecting product strips on a fabric, comprising:
a sensor configured to detect at least one of (a) optical, (b) magnetic and (a) electrical properties of the fabric, the sensor configured to convert a detected property to a measurement signal;
a device configured to establish a signal threshold value; and
a signal device configured to emit at least one of (a) an optical and (b) an acoustical signal when a change in the measurement signal beyond the threshold value is taking place.

2. The measuring device according to claim 1, wherein the fabric is arranged as at least one of (a) a paper-including fabric and (b) a textile-including fabric.

3. The measuring device according to claim 1, wherein the sensor is arranged as a capacitive sensor.

4. The measuring device according to claim 3, wherein the sensor includes a plate-type capacitor device.

5. The measuring device according to claim 4, wherein the plate-type capacitor device includes an open plate-type capacitor.

6. The measuring device according to claim 1, wherein the signal device includes at least one of (a) an illumination device and (b) a loudspeaker.

7. The measuring device according to claim 1, further comprising a display configured to display the threshold value and the measurement signal.

8. The measuring device according to claim 1, further comprising an internal energy supply.

9. The measuring device according to claim 1, further comprising a connection connectable to an energy supply.

10. The measuring device according to claim 1, further comprising a signal output connectable to an external data processing installation.

11. The measuring device according to claim 1, further comprising a USB connection connectable to an energy supply and to an external data processing installation.

12. The measuring device according to claim 1, wherein the measuring device is arranged as a mobile handheld unit.

13. The measuring device according to claim 1, wherein the measuring device is coupleable to a displacement-measurement system.

14. The measuring device according to claim 13, wherein the displacement-measurement system includes a device configured displace the sensor transversely to a longitudinal extension of a product strip over the fabric.

15. The measuring device according to claim 13, wherein the displacement-measurement system includes a position signal output at which a signal is present that is a function of a sensor position.

16. The measuring device according to claim 15, wherein the position signal output is connected to the data processing installation.

17. A system, comprising:
a measurement device configured to detect a product strip on a fabric, the measurement device including:
a sensor configured to detect at least one of (a) optical, (b) magnetic and (c) electrical properties of the fabric, the sensor configured to convert a detected property to a measurement signal;
a device configured to establish a signal threshold value; and
a signal device configured to emit at least one of (a) an optical and (b) an acoustical signal when a change in the measurement signal beyond the threshold value is taking place; and
a displacement-measurement system, the measuring device coupled to the displacement-measurement system.

18. A method for detecting product strips on a fabric, comprising:
detecting at least one of (a) optical, (b) magnetic and (c) electrical properties of the fabric;
converting a detected property to a measurement signal;
establishing a threshold value; and
emitting at least one of (a) an optical and (b) an acoustical signal when a change in the measurement signal beyond the threshold value is taking place.
